SEQUENCE LISTING (4 sequences)

SEQ ID NO: 1 - DNA, 32 bp, Artificial
  PCR Primer
cacaatctagataaaccatattggagggcatc

SEQ ID NO: 2 - DNA, 25 bp, Artificial
  PCR Primer
gggaggaagcttggggagattaatc

SEQ ID NO: 3 - DNA, 1446 bp, Escherichia coli
  DNA fragment (1)..(1446): PCR product
cacaatctagataaaccatattggagggcatcatgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaatctccccaagcttcctccc

SEQ ID NO: 4 - protein, 464 aa, Escherichia coli
MPDAKKQGRSNKAMTFFVCFLAALAGLLFGLDIGVIAGALPFIADEFQITSHTQEWVVSSMMFGAAVGAVGSGWLSFKLGRKKSLMIGAILFVAGSLFSAAAPNVEVLILSRVLLGLAVGVASYTAPLYLSEIAPEKIRGSMISMYQLMITIGILGAYLSDTAFSYTGAWRWMLGVIIIPAILLLIGVFFLPDSPRWFAAKRRFVDAERVLLRLRDTSAEAKRELDEIRESLQVKQSGWALFKENSNFRRAVFLGVLLQVMQQFTGMNVIMYYAPKIFELAGYTNTTEQMWGTVIVGLTNVLATFIAIGLVDRWGRKPTLTLGFLVMAAGMGVLGTMMHIGIHSPSAQYFAIAMLLMFIVGFAMSAGPLIWVLCSEIQPLKGRDFGITCSTATNWIANMIVGATFLTMLNTLGNANTFWVYAALNVLFILLTLWLVPETKHVSLEHIERNLMKGRKLREIGAHD

(19) United States
(12) Patent Application Publication
(10) Pub. No.: US 2004/0214294 A1
(43) Pub. Date: Oct. 28, 2004

(54) Process for the production of L-amino acids using strains of the enterobacteriaceae family

(76) Inventor: Mechthild Rieping, Bielefeld (DE)

Correspondence Address: Michael A. Sanzo, Fitch, Even, Tabin & Flannery, Suite 401L, 1801 K Street, N.W., Washington, DC, 20006-1201, US

(21) Appl. No.: 10/812,315

(22) Filed: Mar. 30, 2004

(30) Foreign Application Priority Data
Apr. 1, 2003 (DE) 103 14 618.0

(51) Int. Cl. C12P 13/04; C12N 1/21; C12N 9/02

(52) U.S. Cl. 435/106; 435/252.33

(57) ABSTRACT

The invention provides a process for the production of L-amino acids, in particular L-threonine, in which the following steps are performed:

a) fermentation of microorganisms from the Enterobacteriaceae family, in which the galP gene or nucleotide sequences coding for the galp gene product are overexpressed and which produce the desired L-amino acid;

b) enrichment of the desired L-amino acid in the medium or in cells of the bacteria; and

c) isolation of the desired L-amino acid.

### FIELD OF THE INVENTION

The present invention provides a process for the production of L-amino acids, in particular L-threonine, using strains of the Enterobacteriaceae family in which the galP gene is overexpressed.

BACKGROUND OF THE INVENTION L-amino acids such as L-threonine are used in human medicine, in the pharmaceutical industry, in the foodstuffs industry and, very particularly, in animal nutrition. It is known that L-amino acids can be prepared by the fermentation of strains of Enterobacteriaceae, especially Escherichia coli (E. coli) and Serratia marcescens. As a result of the great importance of these amino acids, efforts are constantly made to improve production methods. Process improvements may relate to fermentation engineering measures, e.g., methods of stirring and supplying oxygen, or to the composition of the nutrient media, e.g., the sugar concentration present during fermentation. Alternatively, improvements may relate to the way in which product is purified, e.g., ion-exchange chromatography, or to the intrinsic performance characteristics of the microorganism itself

Methods of mutagenesis, selection and mutant choice are often used to improve the performance characteristics of microorganisms. In this way, strains are obtained that are resistant to antimetabolites such as the threonine analog α-amino-β-hydroxyvaleric acid (AHV) or that are auxotrophic for regulatorily important metabolites and which produce L-amino acids such as L-threonine. For some time now, methods of recombinant DNA engineering have also been used for improving L-amino acid-producing strains of the Enterobacteriaceae family. This often involves amplifying individual amino acid biosynthesis genes and testing the effect of this ampification on production. A summary of information relating to the cellular biology and molecular biology of Escherichia coli and Salmonella can be found in Neidhardt (ed.): Escherichia coli and Salmonella, Cellular and Molecular Biology, 2^{nd} edition, ASM Press, Washington, D.C., USA,(1996).

### OBJECT OF THE INVENTION

The object of the present invention is to provide new measures for the improved fermentative production of L-amino acids and, in particular, L-threonine.

### SUMMARY OF THE INVENTION

The invention provides a process for the fermentative production of L-amino acids using microorganisms from the Enterobacteriaceae family in which the nucleotide sequence coding for the galP gene is overexpressed. In addition, the invention encompasses Thus, in its first aspect, the invention is directed to a process for the production of an L-amino acid product by fermenting a microorganism from the Enterobacteriaceae family in a fermentation medium. The microorganism produces the desired L-amino acid and is characterized by increased activity of the galP gene product due to the overexpression of the galP gene or due to the expression of another nucleotide sequence coding for galP. One method for increasing the expression of polynucleotides which code for the galP gene is to increase copy number. Alternatively, expression may be increased by changing the promoter normally found in the galP gene. After allowing the desired amino acid to become enriched in either the fermentation medium or in the microorganism itself, it is isolated to produce the product. The most preferred L-amino acid for production by this process is L-threonine. It is also preferred that some or all of the constituents of the fermentation broth and/or biomass from the microorganism undergoing fermentation remain in the final amino acid product.

The process described above may be carried out using a microorganism in which, in addition to overexpression of galP, at least one gene in a biosynthesis pathway of the L-amino acid being produced is also overexpressed. Examples of specific genes that may be overexpressed include:

a) the thrABC operon coding for aspartate kinase, homoserine dehydrogenase, homoserine kinase and threonine synthase;

b) the pyc gene coding for pyruvate carboxylase;

c) the pps gene coding for phosphoenolpyruvate synthase;

d) the ppc gene coding for phosphoenolpyruvate carboxylase;

e) the pntA and pntB genes coding for transhydrogenase,

f) the rhtB gene which imparts homoserine resistance;

g) the mqo gene coding for malate:quinone oxidoreductase;

h) the rhtC gene which imparts threonine resistance;

i) the thrE gene coding for threonine export protein;

j) the gdhA gene coding for glutamate dehydrogenase;

k) the glk gene coding for glucokinase;

l) the hns gene coding for DNA binding protein HLP-II;

m) the pgm gene coding for phosphoglucomutase,

n) the fba gene coding for fructose biphosphate aldolase;

o) the ptsH gene coding for phosphohistidine protein hexose phospho-transferase;

p) the ptsI gene coding for enzyme I in the phosphotransferase system;

q) the crr gene coding for the glucose-specific IIA component;

r) the ptsG gene coding for the glucose-specific IIBC component;

s) the Irp gene coding for a regulator in the leucine regulon;

t) the csrA gene coding for the global regulator Csr;

u) the fadR gene coding for a regulator in the fad regulon;

v) the icIR gene coding for a regulator in central intermediary metabolism;

w) the mopB gene coding for the 10 KDa chaperone;

x) the ahpC gene coding for the small sub-unit of alkyl hydroperoxide reductase;

y) the ahpF gene coding for the large sub-unit of alkyl hydroperoxide reductase;

z) the cysK gene coding for cysteine synthase A;

aa) the cysB gene coding for the regulator in the cys regulon;

bb) the cysJ gene coding for the flavoprotein in NADPH sulfite reductase;

cc) the cysl gene coding for haemoprotein in NADPH sulfite reductase;

dd) the cysH gene coding for adenylylsulfate reductase;

ee) the phoB gene coding for the positive regulator PhoB in the pho regulon;

ff) the phoR gene coding for the sensor protein in the pho regulon;

gg) the phoE gene coding for protein E in the outer cell membrane;

hh) the pykf gene coding for the pyruvate kinase I stimulated by fructose;

ii) the pfkB gene coding for 6-phosphofructokinase II;

jj) the malE gene coding for periplasmatic binding protein in maltose transport;

kk) the sodA gene coding for superoxidedismutase;

ll) the rseA gene coding for a membrane protein with anti-sigmaE activity;

mm) the rseC gene coding for a global regulator in the sigmae factor;

nn) the sucA gene coding for the decarboxylase sub-unit of 2-ketoglutarate dehydrogenase;

OO) the sucB gene coding for the dihydrolipoyl-transsuccinase E2 subunit of 2-ketoglutarate dehydrogenase;

pp) the sucC gene coding for the β-subunit of succinyl-CoA synthetase;

qq) the sucD gene coding for the α-subunit in succinyl-CoA synthetase;

rr) the adk gene coding for adenylate kinase;

ss) the hdeA gene coding for a periplasmatic protein with a chaperonin-like function;

tt) the hdeB gene coding for a periplasmatic protein with a chaperonin-like function;

uu) the icd gene coding for isocitrate dehydrogenase;

vv) the mglB gene coding for periplasmatic, galactose-binding transport protein;

ww) the lpd gene coding for dihydrolipoamide dehydrogenase;

xx) the aceE gene coding for the El component of pyruvate dehydrogenase complex;

yy) the aceF gene coding for the E2 component of pyruvate dehydrogenase complex;

zz) the pepB gene coding for aminopeptidase B;

aaa) the aldH gene coding for aldehyde dehydrogenase;

bbb) the bfr gene coding for the iron storage homoprotein;

ccc) the udp gene coding for uridine phosphorylase; and

ddd) the rseB gene coding for the regulator of sigmaE factor activity.

Alternatively, a microorganism may be used in which at least one metabolic pathway which reduces the production of the L-amino acid is switched off. Specific genes that may be attenuated by either being switched off or having their expression reduced, include:

a) the tdh gene coding for threonine dehydrogenase;

b) the mdh gene coding for malate dehydrogenase;

c) the gene product of the open reading frame (ORF) yjfA;

d) the gene product of the open reading frame (ORF) ytfP;

e) the pcka gene coding for the enzyme phosphoenol-pyruvate carboxykinase;

f) the poxB gene coding for pyruvate oxidase;

g) the aceA gene coding for isocitrate lyase;

h) the dgsA gene coding for the DgsA regulator in the phosphotransferase system;

i) the fruR gene coding for fructose repressor;

j) the rpoS gene coding for the sigma³⁸-Factor;

k) the aspA gene coding for aspartate ammonium lyase; and

l) the aceB gene coding for malate synthase A gene.

In another aspect, the invention includes a microorganism from the Enterobacteriaceae family, in which the activity of the galP gene product is increased due to either overexpression of the galP gene or due to the expression of other nucleotide sequences coding for galP. Preferably, the microorganism is from the genus Escherichia and produces L-threonine.

### BRIEF DESCRIPTION OF THE FIGURES:

FIG. 1: Map of the plasmid pTrc99AgalP containing the galP gene. Data relating to lengths are to be regarded as approximate. The abbreviations and names used are defined as follows:

Amp: Ampicillin resistance gene

lacI: Gene for the repressor protein in the trc promoters

Ptrc: trc promoter region, IPTG inducible

galP: Coding region of the galP genes

5S: 5S rRNA region

rrnBT: rRNA terminator region

The abbreviations for the restriction enzymes are defined as follows:

BamHI, restriction endonuclease from Bacillus amyloliquefaciens H

EcoRV: restriction endonuclease from Escherichia coli B946

HincII: restriction endonuclease from Haemophilus influence R_{c}

HindIII: restriction endonuclease from Haemophilus influenzae

Kpnl: restriction endonuclease from Klebsiella pneumoniae

Xbal: restriction endonuclease from Xanthomonas badrii (ATTC 11672)

### DEFINITIONS

The gene product encoded by the galp gene is known in the art, inter alia, as “galactose proton symporter” or “galactose permease.”

The protein or ribonucleic acid encoded by a nucleotide sequence, ie., a gene or an allele, is called a gene product.

Alleles are generally understood to be alternative forms of a given gene. The forms are characterized by differences in nucleotide sequence.

Wherever L-amino acids or amino acids are mentioned herein, this is intended to mean one or more amino acids, including their salts, chosen from the group comprising L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophane and L-arginine. L-threonine is particularly preferred.

The word “overexpression,” in this connection, describes the increase in intracellular activity or concentration of one or more enzymes or proteins, in a microorganism, which are coded for by the corresponding DNA. Overexpression may be accomplished, for example, by increasing the copy number of the gene or genes by at least one (1) copy, by using a strong promoter, or by combining these measures.

As a result of overexpression, the activity or concentration of the corresponding protein may be increased by at least 10%, 25%, 50%, 75%, 100%, 150%, 200%, 300%, 400% or 500%, at most up to 1000% or 2000%, with respect to the wild type protein or the activity or concentration of the protein in the starting microorganism. The starting microorganism or parent strain is understood to be the microorganism on which the measures to achieve overexpression are performed.

### DETAILED DESCRIPTION OF THE INVENTION

The invention provides a process for the production of L-amino acids by the fermentation of recombinant microorganisms from the Enterobacteriaceae family in which the galP gene is overexpressed or in which the activity of the galP gene product is increased due to the expression of other sequences coding for this gene product, characterized in that:

a) the microorganisms producing the desired L-amino acid cultivated in a medium under conditions in which the desired L-amino acid is enriched in the medium or in the cells, and

b) the desired L-amino acid is isolated, wherein all or some (≧0 to 100%) of the constituents of the fermentation broth and/or the biomass optionally remain in the isolated product or are completely removed.

The recombinantlt engineered microorganisms are also part of the present invention and can produce L-amino acids from glucose, saccharose, lactose, fructose, maltose, molasses, optionally starch, optionally cellulose or from glycerine and ethanol. They are representatives of the Enterobacteriaceae family chosen from the genera Escherichia, Erwinia, Providencia and Serratia. The genera Escherichia and Serratia are preferred. In the case of the genus Escherichia, the species Escherichia coli is particularly preferred, and in the case of the genus Serratia, the species Serratia marcescens is particularly preferred. Recombinant microorganisms are generally produced by transformation, transduction or conjugation using a vector containing the desired gene.

Suitable strains of the genus Escherichia, in particular species of Escherichia coli producing L-threonine are, for example:

Escherichia coli H4581 (EP-A 0 301 572);

Escherichia coli KY10935 (Bioscience Biotechnology and Biochemistry 61(11):1877-1882 (1997);

Escherichia coli VNIIgenetika MG442 (U.S. Pat. No.4278,765);

Escherichia coli VNIIgenetika M1 (U.S. Pat. No.4,321,325);

Escherichia coli VNIIgenetika 472T23 (U.S. Pat. No.5,631,157);

Escherichia coli BKIIM B-3996 (U.S. Pat. No.5,175,107);

Escherichia coli kat 13 (WO 98/04715);

Escherichia coli KCCM-10132 (WO 00/09660).

Suitable strains of the genus Serratia, in particular species of Serratia marcescens producing L-threonine are, for example:

Serratia marcescens HNr21 (Applied and Environmental Microbiology 38(6): 1045-1051 (1979));

Serratia marcescens TLrl56 (Gene 57(2-3): 151-158 (1987));

Serratia marcescens T-2000 (Applied Biochemistry and Biotechnology 37(3): 255-265 (1992)).

L-threonine-producing strains from the Enterobacteriaceae family preferably possess, inter alia, one or more of the genetic or phenotypical features chosen from the group: resistance to α-amino-β-hydroxyvaleric acid, resistance to thialysine, resistance to ethionine, resistance to α-methylserine, resistance to diaminosuccinic acid, resistance to α-aminobutyric acid, resistance to borrelidine, resistance to cyclopentanecarboxylic acid, resistance to rifampicin, resistance to valine analogues such as, for example, valinehydroxamate, resistance to purine analogues such as, for example, 6-dimethyl-aminopurine, a requirement for L-methionine, optionally a partial and compensable requirement for L-isoleucine, a requirement for meso-diaminopimelic acid, auxotrophy with respect to threonine-containing dipeptides, resistance to L-threonine, resistance to threonine raffinate, resistance to L-homoserine, resistance to L-lysine, resistance to L-methionine, resistance to L-glutamic acid, resistance to L-aspartate, resistance to L-leucine, resistance to L-phenylalanine, resistance to L-serine, resistance to L-cysteine, resistance to L-valine, sensitivity towards fluoropyruvate, defective threonine dehydrogenase, optionally the ability to utilize saccharose, overexpression of the threonine operon, overexpression of homoserine dehydrogenase I-aspartate kinase I, preferably the feedback resistant form, overexpression of homoserine kinase, overexpression of threonine synthase, overexpression of aspartate kinase, optionally the feedback resistant form, overexpression of aspartate semialdehyde dehydrogenase, overexpression of phosphoenolpyruvate carboxylase, optionally the feedback resistant form, overexpression of phosphoenolpyruvate synthase, overexpression of transhydrogenase, overexpression of the RhtB gene product, overexpression of the RhtC gene product, overexpression of the YfiK gene product, overexpression of a pyruvate carboxylase, and attenuation of acetic acid formation.

It was found that microorganisms of the Enterobacteriaceae family produce L-amino acids, in particular L-threonine, in an improved manner after overexpression of the galP gene. The nucleotide sequences of the genes from Escherichia coli are part of the prior art and can be obtained from the genome sequence for Escherichia coli published by Blattner et al. (Science 277:1453-1462 (1997)). The galP gene is characterized, inter alia, by the following:

Name: sugar transporter, galactose-proton symporter;

Function: as an integral membrane protein, symport of 2-deoxy-D-galactose and a proton into cells;

References: Macpherson et al.; Journal of Biological Chemistry 258(7): 4390-4396 (1983), Venter et al.; Biochemical Journal 363(Pt 2): 243-252 (2002); Accession No.: AE000377

Galactose permease in Salmonella typhimurium is described, inter alia, in: Postma P. W.; J. Bacteriol. 129(2): 630-639 (1977); and Nagelkerke and Postma, J. Bacteriol. 133(2): 607-613 (1978).

The nucleic acid sequences can be obtained from the databank at the National Center for Biotechnology Information (NCBI) at the National Library of Medicine (Bethesda, Md., USA), the nucleotide sequence databank at the European Molecular Biology Laboratory (EMBL, Heidelberg, Germany and Cambridge, UK) or from the DNA databank of Japan (DDBJ, Mishima, Japan). For the sake of better clarity, the nucleotide sequence of the galP gene from Escherichia coli is given as SEQ ID NO:3 and the amino acid sequence of the galactose-proton symporter protein coded by this gene is given as SEQ ID NO:4.

The genes described in the cited literature references can be used in accordance with the invention. Furthermore, alleles of the genes can be used where these are produced by degeneracy of the genetic code or by functionally neutral sense mutations. The use of endogenous genes is preferred. “Endogenous genes” or “endogenous nucleotide sequences” are understood to be the genes or alleles or nucleotide sequences present in the population of a species.

Included among the alleles which contain functionally neutral sense mutations are those which lead to a conservative amino acid exchange in the protein coded by them. In the case of aromatic amino acids, conservative exchanges are referred to when phenylalanine, tryptophan and tyrosine replace each other. In the case of hydrophobic amino acids, conservative exchanges are referred to when leucine, isoleucine and valine replace each other. In the case of polar amino acids, conservative exchanges are referred to when glutamine and asparagine replace each other. In the case of basic amino acids, conservative exchanges are referred to when arginine, lysine and histidine replace each other. In the case of acidic amino acids, conservative exchanges are referred to. when aspartic acid and glutamic acid replace each other. In the case of hydroxyl group-containing amino acids, conservative exchanges are referred to when serine and threonine replace each other.

In the same way, nucleotide sequences can be used which code for variants of the proteins mentioned which, in addition, are lengthened or shortened at the N- or C-terminal by at least one (1) amino acid. This extension or reduction should not be more than 50, 40, 30, 20, 10, 5, 3 or 2 amino acids or amino acid groupings.

In order to overexpress a gene product, the copy number of the corresponding gene can be increased. Alternatively, the promoter and regulation region or the ribosome binding site can be mutated. Expression cassettes which are incorporated upstream of the structural gene act in the same way. Included among the promoters which can be used are, inter alia, promoters of the lactose and tryptophan operons of Escherichia coli which are known as “lac” and “trp” promoters. The hybrid promoter, “tac,” may also be used (DeBoer et al.; Proc. Nat'l Acad. Sci. USA 80:21-25 (1983)). Other promoters which can be used are the left-directed P_{L} promoter of the lambda bacteriophages, and promoters of the T7 phages, which interact with a repressor, as do the lac, trp and tac promoters already mentioned. By using inducible promoters, it is also possible to increase expression during the course of fermentative L-threonine production. Expression is also improved by measures that prolong the lifetime of m-RNA. In addiiton, enzyme activity may be increased by preventing degradation of the enzyme protein. Genes or gene constructs may either be present in plasmids with different copy numbers or be integrated and amplified in the chromosome. Alternatively, overexpression of the relevant genes may be achieved by modifying the composition of the medium and culture management. A person skilled in the art can find instructions for this, inter alia, in: Chang, et al., J. Bacteriol. 134:1141-1156 (1978); Hartley, et al., Gene 13:347-353 (1981); Amann, et al., Gene 40:183-190 (1985); de Broer, et al., Proc. Nat'l Acad. Sci. USA 80:21-25 (1983); LaVallie, et al., BIO/TECHNOLOGY11:187-193 (1993); W098/04715; Llosa, et al. Plasmid 26:222-224 (1991); Quandt, et al., Gene 80: 161-169 (1989); Hamilton, et al., J. Bacteriol. 171:4617-4622 (1989); Jensen, et al., Biotechnol. Bioeng. 58:191-195 (1998)); and in well-known textbooks on genetics and molecular biology.

Plasmid vectors which can be replicated in Enterobacteriaceae, include, e.g., cloning vectors derived from pACYCi84 (Bartolome et al.; Gene 102:75-78 (1991)); pTrc99A (Amann et al.; Gene 69:301-315 (1988) and pSC101-derivates (Vocke, et al., Proc. Nat'l Acad. Sci. USA 80(21):6557-6561 (1983)). In a process according to the invention, a strain transformed with a plasmid vector may be used, wherein the plasmid vector contains at least one nucleotide sequence coding for the galP gene. The expression “transformation” is understood to be the acceptance of an isolated nucleic acid by a host (microorganism). It is also possible to introduce mutations which affect expression of a gene by sequence exchange (Hamilton, et al.; J. Bacteriol. 171: 4617-4622 (1989)), conjugation or transduction in different strains. More detailed explanations of the concepts used in genetics and molecular biology can be found in well-known textbooks of genetics and molecular biology, such as: Birge, Bacterial and Bacteriophage Genetics, 4^{th} ed., Springer Verlag, New York, USA, 2000; Berg, Tymoczko and Stryer, Biochemistry, 5^{th} ed., Freeman and Company, New York, USA, 2002), Sambrook, et al., Molecular Cloning. A Laboratory Manual, 3-Volume Set, Cold Spring Harbor Laboratory Press, Cold Spring Harbor, USA, 2001.

It may be advantageous for the production of L-amino acids, in particular L-threonine, using strains of the Enterobacteriaceae family, in addition to overexpressing the galP gene, to overexpress one or more enzymes in the well-known threonine biosynthesis pathway, enzymes from anoplerotic metabolism, enzymes for the production of reduced nicotinamide-adenine dinucleotide phosphate, enzymes from glycolysis, PTS enzymes, or enzymes from sulfur metabolism. The use of endogeneous genes is generally preferred. Thus, for example, one or more genes may be overexpressed from the group:

the thrABC operon coding for aspartate kinase, homoserine dehydrogenase, homoserine kinase and threonine synthase (U.S. Pat. No. 4,278,765);

the pyc gene from Corynebacterium glutamicum coding for pyruvate carboxylase (WO 99/18228);

the pps gene coding for phosphoenolpyruvate synthase (Mol. Gen. Genetics 231(2):332-336 (1992));

theppc gene coding for phosphoenolpyruvate carboxylase (Gene 31:279-283 (1984));

the pntA and pntB genes coding for transhydrogenase (Eur. J Biochem. 158: 647-653 (1986));

the rhtB gene which imparts homoserine resistance (EP-A-0 994 190);

the mqo gene coding for malate:quinone oxidoreductase (WO 02/06459);

the rhtC gene which imparts threonine resistance (EP-A-1 013 765);

the thrE gene from Corynebacterium glutamicum coding for threonine export protein (WO 01/92545);

the gdhA gene coding for glutamate dehydrogenase (Nucl. Ac. Res. 11: 5257-5266 (1983); Gene 23:199-209 (1983));

the glk gene coding for glucokinase (J. Bacteriol 179:1298-1306 (1997));

the hns gene coding for DNA binding protein HLP-II (WO 03/004671);

the pgm gene coding for phosphoglucomutase (WO 03/004598);

the fba gene coding for fructose biphosphate aldolase (WO 03/004664);

the ptsH gene from the ptsfHIcrr operon coding for phosphohistidine protein hexose phosphotransferase in the phosphotransferase system PTS (WO 03/004674);

the ptsl gene from the ptsFHIcrr operon coding for enzyme I in the phosphotransferase system PTS (WO 03/004674);

the crr gene from the ptsHIcrr operon coding for the glucose-specific IIA component in the phosphotransferase systems PTS (WO 03/004674);

the ptsG gene coding for the glucose-specific IIBC component (WO 03/004670);

the Irp gene coding for the regulator in the leucine regulon (WO 03/004665);

the csrA gene coding for the global regulator Csr (J. Bacteriol 1 75:4744-4755 (1993);

the fadR gene coding for the regulator in the fad regulon (Nucl. Ac. Res. 16:7995-8009 (1988));

the iclR gene coding for the regulator in the central intermediary metabolism (J. Bacteriol. 172: 2642-2649 (1990)),

the mopB gene coding for the 10 KDa chaperone (WO 03/004669), which is also known under the name groES;

the ahpC gene from the ahpCF operon coding for the small sub-unit of alkyl hydroperoxide reductase (WO 03/004663);

the ahpF gene from the ahpCF operon coding for the large sub-unit of alkyl hydroperoxide reductase (WO 03/004663);

the cysK gene coding for cysteine synthase A (WO 03/006666);

the cysB gene coding for the regulator in the cys regulon (WO 03/006666);

the cysJ gene from the cysJIH operon coding for the flavoprotein in NADPH sulfite reductase (WO 03/006666);

the cysl gene from the cysJIH operon coding for haemoprotein in NADPH sulfite reductase (WO 03/006666);

the cysH gene from the cysJIH operon coding for adenylylsulfate reductase (WO 03/006666);

the phoB gene from the phoBR operon coding for the positive regulator PhoB in the pho regulon (WO 03/008606);

the phoR gene from the phoBR operon coding for the sensor protein in the pho regulon (WO 03/008606);

the phoE gene coding for protein E in the outer cell membrane (WO 03/008608);

the pykF gene coding for the pyruvate kinase I stimulated by fructose (WO 03/008609);

the pfkB gene coding for 6-phosphofructokinase II (WO 03/008610);

the malE gene coding for periplasmatic binding protein in maltose transport (WO 03/008605);

the sodA gene coding for superoxidedismutase (WO 03/008613);

the rseA gene from the rseABC operon coding for a membrane protein with anti-sigmaE activity (WO 03/008612);

the rseC gene from the rseABC operon coding for a global regulator in the sigmaE factor (WO 03/008612);

the sucA gene from the sucABCD operon coding for the decarboxylase sub-unit of 2-ketoglutarate dehydrogenase (WO 03/008614);

the sucB gene from the sucABCD operon coding for the dihydrolipoyltranssuccinase E2 sub-unit of 2-ketoglutarate dehydrogenase (WO 03/008614);

the sucC gene from the sucABCD operon coding for the β-sub-unit of succinyl-CoA synthetase (WO 03/008615);

the sucD gene from the sucABCD operon coding for the α-sub-unit in succinyl-CoA synthetase (WO 03/008615);

the adk gene coding for adenylate kinase (Nucl. Ac. Res. 13(19):7139-7151 (1985));

the hdeA gene coding for a periplasmatic protein with a chaperonin-like function (J. Bacteriol. 175(23): 7747-7748 (1993)),

the hdeB gene coding for a periplasmatic protein with a chaperonin-like function (J. Bacteriol. 175(23): 7747-7748 (1993));

the icd gene coding for isocitrate dehydrogenase (J. Biol. Chem. 262(22): 10422-10425 (1987));

the mglB gene coding for periplasmatic, galactose-binding transport protein (Mol. Gen. Genet. 229(3):453-459 (1991));

the lpd gene coding for dihydrolipoamide dehydrogenase (Eur. J. Biochem. 135(3): 519-527 (1983));

the aceE gene coding for the E1 component of pyruvate dehydrogenase complex (Eur. J. Biochem. 133(1): 155-162 (1983));

the aceF gene coding for the E2 component of pyruvate dehydrogenase complex (Eur. J Biochem. 133(3):481-489 (1983));

the pepB gene coding for aminopeptidase B (J. Ferment. Bioengin. 82:392-397 (1996));

the aldH gene coding for aldehyde dehydrogenase (E.C. 1.2.1.3) (Gene 99(1): 15-23 (1991));

the bfr gene coding for the iron storage homoprotein (bacterioferritin) (J. Bacteriology 171(7): 3940-3947 (1989));

the udp gene coding for uridine phosphorylase (Nucl. Ac.Res. 17(16): 6741 (1989)); and

the rseB gene coding for the regulator of sigmaE factor activity (Mol. Microbiol. 24(2): 355-371 (1997)).

It may also be advantageous for the production of L-amino acids, in particular L-threonine, in addition to overexpressing the galP gene, to attenuate, (switch off or reduce the expression of), one or more genes chosen from the group:

the tdh gene coding for threonine dehydrogenase (J Bacteriol. 169:4716-4721 (1987));

the mdh gene coding for malate dehydrogenase (E.C. 1.1.1.37) (Arch. Microbiol. 149:36-42 (1987));

the gene product of the open reading frame (ORF) yjfA (Accession Number AAC77180 at the National Center for Biotechnology Information, NCBI, Bethesda, Md., USA, WO 02/29080);

the gene product of the open reading frame (ORF) ytfp (Accession Number AAC77179 at the National Center for Biotechnology Information (NCBI, Bethesda, Md., USA, WO 02/29080);

the pcka gene coding for the enzyme phosphoenolpyruvate carboxykinase (WO 02/29080);

the poxB gene coding for pyruvate oxidase (WO 02/36797);

the aceA gene coding for the enzyme isocitrate lyase (WO 02/081722);

the dgsA gene coding for the DgsA regulator in the phosphotransferase system (WO 02/081721), which is also known as the mlc gene;

the fruR gene coding for fructose repressor (WO 02/081698), which is also known as the cra gene;

the rpoS gene coding for the sigma³⁸-Factor (WO 01/05939), which is also known as the katF gene;

the aspA gene coding for aspartate ammonium lyase (WO 03/008603); and

the aceB gene coding for malate synthase A (WO 03/008603).

The expression “attenuation” in this connection describes the reduction in or switching off of intracellular activity or concentration of one or more enzymes or proteins in a microorganism by, for example: using a weak promoter; using a gene or allele which codes for a corresponding enzyme or protein with a lower activity; or by inactivating the corresponding enzyme or protein or gene; and optionally by combining these measures. Due to attenuation, the activity or concentration of the corresponding protein is generally lowered to 0 to 75%, 0 to 50%, 0 to 25%, 0 to 10% or 0 to 5% of the activity or concentration of the wild-type protein in the starting microorganism. Furthermore, it may be advantageous for the production of L-amino acids, in particular L-threonine, in addition to overexpressing the galp gene, to switch off undesired side reactions (Nakayama: “Breeding of Amino Acid Producing Microorganisms”, in: Overproduction of Microbial Products, Krumphanzl, Sikyta, Vanek (eds.), Academic Press, London, UK, 1982).

Microorganisms produced in accordance with the invention may be cultivated in a batch process, in a fed batch process or in a repeated fed batch process. A summary of known cultivation methods is given in the textbook by Chmiel (Bioprozesstechnik1. Einfürung in die Bioverfahrenstechnik, Gustav Fischer Verlag, Stuttgart, 1991) or in the textbook by Storhas (Bioreaktoren und periphere Einrichtungen (Vieweg Verlag, Braunschweig/Wiesbaden, 1994)).

The culture medium to be used must satisfy the demands of the particular bacterial striain baing used strain in an appropriate manner. Descriptions of culture media for different microorganisms are given in the manual “Manual of Methods for General Bacteriology” by the American Society for Bacteriology (Washington D.C., USA, 1981).

Suitable sources of carbon which may be used are sugar and carbohydrates such as e.g. glucose, saccharose, lactose, fructose, maltose, molasses, starch and optionally cellulose, oils and fats such as e.g. soy oil, sunflower oil, ground nut oil and coconut fat, fatty acids such as palmitic acid, stearic acid and linoleic acid, alcohols such as e.g. glycerine and ethanol and organic acids such as e.g. acetic acid. These substances may be used separately or as a mixture.

Sources of nitrogen which may be used are organic nitrogen-containing compounds such as peptones, yeast extract, meat extract, malt extract, corn steep water, soy bean flour and urea or inorganic compounds such as ammonium sulfate, ammonium chloride, ammonium phosphate, ammonium carbonate and ammonium nitrate. The sources of nitrogen may be used separately or as a mixture.

Sources of phosphorus which may be used are phosphoric acid, potassium dihydrogen phosphate or dipotassium hydrogen phosphate or the corresponding sodium-containing salts. The culture medium also must contain salts of metals such as magnesium sulfate or iron sulfate which are needed for growth. Finally, essential growth substances such as amino acids and vitamins may also be used in addition to the substances mentioned above. Suitable precursors may also be added to the culture medium. The feedstocks mentioned above may be added to the culture in the form of a single mixture or may be fed during cultivation in an appropriate manner.

Fermentation is generally performed at a pH of 5.5 to 9.0, and preferably at 6.0 to 8.0. Basic compounds such as sodium hydroxide, potassium hydroxide, ammonia and ammonia water or acid compounds such as phosphoric acid or sulfuric acid are used in an appropriate manner to control the pH Anti-foam agents such as e.g., fatty acid polyglycol esters can be used to control the formation of foam. Substances which act in a selective manner, such as antibiotics, can be added to the medium to maintain stability of the plasmids. In order to maintain aerobic conditions, oxygen or oxygen-containing gases, such as air, are introduced to the culture. The temperature of the culture is normally 25° C. to 45° C. and preferably 30° C. to 40° C. The culture is continued until a maximum of L-amino acids or L-threonine has been produced. This objective is normally achieved within 10 hours to 160 hours.

Analysis of L-amino acids can be performed by anion exchange chromatography followed by ninhydrin derivation, as is described in Spackman et al., Anal. Chem. 30: 1190-1206 (1958)), or it can be performed by reversed phase HPLC, as is described in Lindroth et al. (Anal. Chem. 51:1167-1174 (1979)). The process according to the invention is used for the fermentative production of L-amino acids such as, for example, L-threonine, L-isoleucine, L-valine, L-methionine, L-homoserine and L-lysine, in particular L-threonine.

The present invention is explained in more detail in the following working examples. The minimal (M9) and full medium (LB) for Escherichia coli are described by J. H. Miller (A Short Course in Bacterial Genetics (1992), Cold Spring Harbor Laboratory Press). Isolation of plasmid DNA from Escherichia coli and all techniques relating to restriction, ligation, Klenow and alkaline phosphatase treatments are performed as described by Sambrook et al. (Molecular Cloning—A Laboratorv Manual (1989), Cold Spring Harbor Laboratory Press). Transformation of Escherichia coli is performed, unless described otherwise, as described by Chung et aL (Proc. Nat'l Acad. Sci. USA 86: 2172-2175 (1989)). The incubation temperature during the production of strains and transformants is 37° C.

### EXAMPLES

### Example 1

### Construction of the Expression Plasmid pTrc99AgalP

The galP gene from E. coli K12 is amplified using the polymerase chain reaction (PCR) and synthetic oligonucleotides. Starting from the nucleotide sequence of the galP gene in E. coli K12 MG1655 (Accession Number AE000377, Blattner et al., Science 277:1453-1474 (1997)), PCR primers are synthesized. (MWG Biotech, Ebersberg, Germany). The sequences of the primer are modified so that recognition sites for restriction enzymes are produced. The recognition sequence for XbaI is chosen for the galPi primer and the recognition site for HindIlI is chosen for the galP2 primer, these being indicated by underlining in the nucleotide sequences shown below:

| | |
|---|---|
| galP1: | |
| 5′-CACAATCTAGATAAACCATATTGGAGGG | (SEQ ID NO: 1) |
| CATC-3′ | |
| galP2: | |
| 5′-GGGAGGAAGCTTGGGGAGATTAATC-3′ | (SEQ ID NO: 2) |

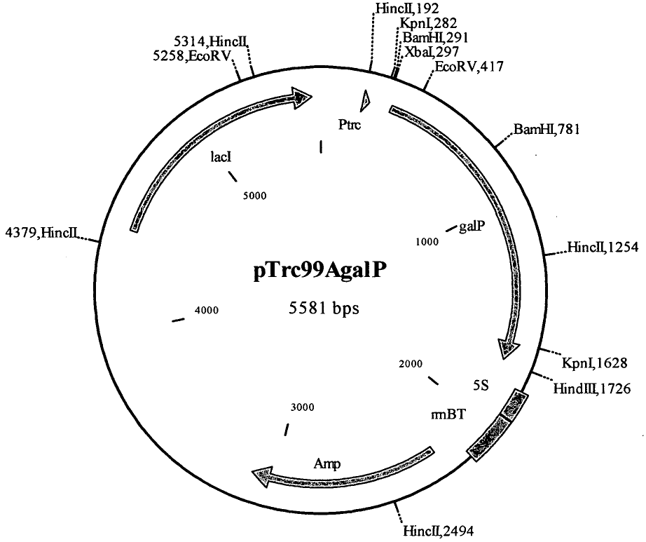

The chromosomal E. coli K12 MG1655 DNA used for PCR is isolated in accordance with the manufacturer's instructions, using “Qiagen Genomic-tips 100/G” (QIAGEN, Hilden, Germany). An approximately 1450 bp sized DNA fragment can be amplified with the specific primers under standard PCR conditions (Innis et al. (1990) PCR Protocols. A Guide to Methods and Applications, Academic Press) using Vent DNA polymerase (New England Biolabs GmbH, Frankfurt, Germany) (SEQ ID NO:3). The amplified galP fragment is cleaved with the enzymes XbaI and HindIII and ligated with the vector pTrc99A (Pharmacia Biotech, Uppsala, Sweden) which has been digested with the enzymes XbaI and HindIII. The E. coli strain TOP 10 One Shot® (TOPO TA Cloning Kit, Invitrogen, Groningen, Netherlands) is transformed with the ligation mixture and plasmid-containing cells are selected on LB agar which has been treated with 50 μg/ml ampicillin. Successful cloning can be detected after plasmid DNA isolation by test cleavage with the enzymes XbaI, HindIII and EcoRV. The plasmid is called pTrc99AgalP (FIG. 1).

### Example 2

### Production of L-threonine with the strain MG442/pTrc99AgalP

The L-threonine producing E. coli strain MG442 is described in patent specification U.S. Pat. No.4,278,765 and is deposited in the Russian National Collection of Industrial Microorganisms (VKPM, Moscow, Russia) as CMIM B-1628. The strain MG442 is transformed with the expression plasmid pTrc99AgalP described in example 1 and with the vector pTrc99A. Plasmid-containing cells are selected on LB agar with 50 μg/ml ampicillin. Successful transformation can be confirmed after plasmid DNA isolation by test cleavages with the enzymes HincII, BamHI and Kpnl. The strains MG442/pTrc99AgalP and MG442/pTrc99A are produced in this way.

Selected individual colonies are then multiplied again on minimal medium with the following composition: 3.5 g/l Na₂HPO₄*2H₂O, 1.5 g/l KH₂PO₄, 1 g/l NH₄Cl, 0.1 g/l MgSO₄*7H₂O, 2 g/l glucose, 20 g/l agar, 50 mg/l ampicillin. The production of L-threonine is checked in batch cultures of 10 ml which are placed in 100 ml Erlenmeyer flasks. 10 ml preculture medium with the following composition is added to these: 2 g/l yeast extract, 10 g/l (NH₄)₂SO₄, 1 g/l KH₂PO₄, 0.5 g/l MgSO₄*7H₂0, 15 g/l CaCO₃, 20 g/l glucose, 50 mg/l ampicillin, inoculated and incubated for 16 hours at 37° C. and 180 rpm on an ESR incubator from Kühner AG (Birsfelden, Switzerland). 250 μl of each of these precultures are then inoculated into 10 ml of production medium (25 g/l (NH₄)₂SO₄, 2 g/l KH₂PO₄, 1 g/l MgSO₄*7H₂O, 0.03 g/l FeSO₄*7H₂O, 0.018g/l MnSO₄*1H₂O, 30 g/l CaCO₃, 20g/l glucose, 50 mg/l ampicillin) and incubated for 48 hours at 37° C. The production of L-threonine by the starting strain MG442 is checked in the same way, except that no ampicillin is added to the medium. After incubation, the optical density (OD) of the culture suspension is determined at a measurement wavelength of 660 nm using a LP2W photometer from the Dr. Lange Co. (Düsseldorf, Germany).

Finally, the concentration of L-threonine produced in sterile filtered culture supernatant liquid is determined using an amino acid analyzer from Eppendorf-BioTronik (Hamburg, Germany) by means of ion exchange chromatography and post-column reaction with ninhydrin detection. Table 1 gives the results of the trial.

TABLE 1

| | OD | |
|---|---|---|
| Strain | (660 nm) | L-threonine g/l |
| MG442 | 5.6 | 1.4 |
| MG442/pTrc99A | 3 | 1.3 |
| MG442/pTrc99AgalP | 4.1 | 1.9 |

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof

What is claimed is:

1. A process for the production of an L-amino acid product comprising:
a) fermenting a microorganism from the Enterobacteriaceae family in a fermentation medium, wherein said microorganism produces said L-amino acid and wherein the galP gene is overexpressed or another nucleotide sequence coding for galP is expressed in said microorganism;
b) enriching said L-amino acid in said fermentation medium or in said microorganism; and
c) isolating said L-amino acid to produce said amino acid product.

2. The process of claim 1, wherein said L-amino acid is L-threonine.

3. The process of either claim 1 or claim 2, wherein some or all of the constituents of said fermentation medium and/or the biomass of said microorganism remain in said amino acid product.

4. The process of either claim 1 or claim 2, wherein at least one gene in the biosynthesis pathway of said L-amino acid is overexpressed.

5. The process of either claim 1 or claim 2, wherein at least one metabolic pathway which reduces the production of said L-amino acid in said microorganism is switched off.

6. The process of either claim 1 or claim 2, wherein expression of a polynucleotide which codes for the galP gene has been increased by increasing the copy number of the gene.

7. The process of either claim 1 or claim 2, wherein expression of the galP gene in said microorganism has been increased by changing the promoter normally found in said galP gene.

8. The process of either claim 1 or claim 2, wherein said microorganism overexpresses one or more genes selected from the group consisting of:
a) the thrABC operon coding for aspartate kinase, homoserine dehydrogenase, homoserine kinase and threonine synthase;
b) the pyc gene coding for pyruvate carboxylase;
c) the pps gene coding for phosphoenolpyruvate synthase;
d) the ppc gene coding for phosphoenolpyruvate carboxylase;
e) the pnta and pntb genes coding for transhydrogenase,
f) the rhtb gene which imparts homoserine resistance;
g) the mqo gene coding for malate:quinone oxidoreductase;
h) the rhtC gene which imparts threonine resistance;
i) the thrE gene coding for threonine export protein;
j) the gdhA gene coding for glutamate dehydrogenase;
k) the glk gene coding for glucokinase;
l) the hns gene coding for DNA binding protein HLP-II;
m) the pgm gene coding for phosphoglucomutase;
n) the fba gene coding for fructose biphosphate aldolase;
o) the ptsH gene coding for phosphohistidine protein hexose phosphotransferase;
p) the ptsI gene coding for enzyme I in the phosphotransferase system;
q) the crr gene coding for the glucose-specific IIA component;
r) the ptsG gene coding for the glucose-specific IIBC component;
s) the lrp gene coding for a regulator in the leucine regulon;
t) the csrA gene coding for the global regulator Csr;
u) the fadR gene coding for a regulator in the fad regulon;
v) the iclR gene coding for a regulator in central intermediary metabolism;
w) the mopB gene coding for the 10 KDa chaperone;
x) the ahpC gene coding for the small sub-unit of alkyl hydroperoxide reductase;
y) the ahpF gene coding for the large sub-unit of alkyl hydroperoxide reductase;
z) the cysK gene coding for cysteine synthase A;
aa) the cysB gene coding for the regulator in the cys regulon;
bb) the cysJ gene coding for the flavoprotein in NADPH sulfite reductase;
cc) the cysI gene coding for haemoprotein in NADPH sulfite reductase;
dd) the cysH gene coding for adenylylsulfate reductase;
ee) the phoB gene coding for the positive regulator PhoB in the pho regulon;
ff) the phoR gene coding for the sensor protein in the pho regulon;
gg) the phoE gene coding for protein E in the outer cell membrane;
hh) the pykF gene coding for the pyruvate kinase I stimulated by fructose;
ii) the pfkB gene coding for 6-phosphofructokinase II;
jj) the malE gene coding for periplasmatic binding protein in maltose transport;
kk) the sodA gene coding for superoxidedismutase;
ll) the rseA gene coding for a membrane protein with anti-sigmaE activity;
mm) the rseC gene coding for a global regulator in the sigmaE factor;
nn) the sucA gene coding for the decarboxylase sub-unit of 2-ketoglutarate dehydrogenase;
oo) the sucB gene coding for the dihydrolipoyl-transsuccinase E2 subunit of 2-ketoglutarate dehydrogenase;
pp) the sucC gene coding for the β-subunit of succinyl-CoA synthetase;
qq) the sucD gene coding for the α-subunit in succinyl-CoA synthetase;
rr) the adk gene coding for adenylate kinase;
ss) the hdeA gene coding for a periplasmatic protein with a chaperonin-like function;
tt) the hdeB gene coding for a periplasmatic protein with a chaperonin-like function;
uu) the icd gene coding for isocitrate dehydrogenase;
vv) the mglB gene coding for periplasmatic, galactose-binding transport protein;
ww) the lpd gene coding for dihydrolipoamide dehydrogenase;
xx) the aceE gene coding for the E1 component of pyruvate dehydrogenase complex;
yy) the aceF gene coding for the E2 component of pyruvate dehydrogenase complex;
zz) the pepB gene coding for aminopeptidase B;
aaa) the aldH gene coding for aldehyde dehydrogenase;
bbb) the bfr gene coding for the iron storage homoprotein;
ccc) the udp gene coding for uridine phosphorylase; and
ddd) the rseB gene coding for the regulator of sigmae factor activity.

9. The process of either claim 1 or claim 2, wherein at least one gene in said microorganism is attenuated by either being switched off or having its expression reduced, said gene being selected from the group consisting of:
a) the tdh gene coding for threonine dehydrogenase;
b) the mdh gene coding for malate dehydrogenase;
c) the gene product of the open reading frame (ORF) yjfA;
d) the gene product of the open reading frame (ORF) ytfp;
e) the pckA gene coding for the enzyme phosphoenol-pyruvate carboxykinase;
f) the poxB gene coding for pyruvate oxidase;
g) the aceA gene coding for isocitrate lyase;
h) the dgsA gene coding for the DgsA regulator in the phosphotransferase system;
i) the fruR gene coding for fructose repressor;
j) the rpoS gene coding for the sigma³⁸-Factor;
k) the aspA gene coding for aspartate ammonium lyase; and
l) the aceB gene coding for malate synthase A gene.

10. A microorganism from the Enterobacteriaceae family, in which the galP gene or or other nucleotide sequences coding for galP are overexpressed.

11. The microorganism of claim 10, wherein said microorganism is from the genus Escherichia.

12. The microorganism of either claim 10 or claim 11 wherein said microorganism produces L-threonine.
